(19) United States
(12) Patent Application Publication
(10) Pub. No.: US 2006/0178340 A1
(43) Pub. Date: Aug. 10, 2006

(54) Composition comprising soluble glucan oligomer from saccharomyces cerevisiae is2 for immune activation or prevention and treatment of cancer and the preparation method thereof

(76) Inventor: Bong-Hyun Chung, Taejeon (KR)

Correspondence Address: KIRK HAHN, 14431 HOLT AVE, SANTA ANA, CA, 92705, US

(21) Appl. No.: 10/549,016

(22) PCT Filed: Mar. 17, 2004
(86) PCT No.: PCT/KR04/00584

§ 371 Date: Sep. 14, 2005

(30) Foreign Application Priority Data
Mar. 18, 2003 (KR) 10-2003-0016665

(51) Int. Cl. A61K 31/716; C12P 19/04

(52) U.S. Cl. 514/54; 435/101

(57) ABSTRACT

The present invention relates to the soluble glucan oligomer having a M.W. ranging from 1,000 to 10,000 prepared by treating insoluble beta-glucan isolated the cell wall of yeast variant IS2 with commercially available beta-glucan hydrolyzing enzymes. The soluble glucan oligomer showed potent efficacy on promoting immune activity and on inhibiting the growth of cancer cell, therefore, it can be used as the therapeutics or health care food for treating and preventing immunodeficiency and cancer disease.

### TECHNICAL FIELD

The present invention relates to the composition comprising soluble glucan oligomer isolated from Saccharomyces cerevisiae IS2 for immune activation or prevention and treatment of cancer, and the preparation method thereof.

### BACKGROUND ART

Beta-glucan can be isolated from various resources such as yeast, microorganism, mushroom, grain and algae. It has been studied and applied as various types of product till now. In particular, beta-glucan derived from yeast cell wall has been studied and known well.

Yeast, a microorganism classified into GRAS (Generally Recognized As Safe) in FDA, has been used in various field including food field and the inner cell membrane of yeast comprises beta 1,3- and 1,6-glucan as main ingredients, and a small amount of chitin and mannoprotein, however, outer cell membrane thereof comprises mannoprotein, a protein linked to mannan.

Beta-glucan, a major component of yeast cell wall, has been reported to increase Ag-specific immune response by activation and proliferation of macrophage, to elevate the resistance to pathogen such as fungi, bacteria, virus and the like, to inhibit the immune depression observed in trauma and to increase resistance to cancer or cancer metastasis in a host (Abel, G. and Czop, J. K., Int. J. Immunophamacol., 14, pp 1363-1373, 1992; Babineau, et al., 220(5). Pp 601-609, 1994; Benach J. L., et al., Infection and Immunity, 35(3), pp 947-951, 1982; Di Renzo, L., et al., Eur. J. Immunol., 21, pp 1755-1758, 1991; Fukase, S., et al., Cancer Res., 47, pp 4842-4847, 1987; Janusz, M. J., et al., J. Immun., 142, pp 959-965, 1989; Olsen, E, J., et al., J. Immun., 64, pp 3548-3554, 1996, Sakurai, T., et al., Int. J. Immunopharmacol., 14, pp 821-830, 1992; Czop, J. K., et al., Prog. Clin. Biol. Res., 297, pp287-296, 1989).

Since beta-glucan of yeast is a water-insoluble polysaccharide, a number of preparation methods to obtain beta-glucan with high solubility have been developed till now as follows.

U.S. Pat. No. 5,576,015 discloses the method of preparing beta-glucan with a form of fine particle to increase its absorption rate; U.S. Pat. No. 4,877,777 discloses the method of introducing chemical formula into glucan to increase its solubility; U.S. Pat. No. 5,037,972 and U.S. Pat. No. 6,143,883 disclose the method of preparing soluble glucan particles by extracting glucan with organic solvent and subsequently treating with beta-glucanase or cellulase which can degrade beta-1,3-D-glucose chain, a basic structure of the glucan.

However, there has been not reported or disclosed about the specific soluble glucan oligomer isolated from yeast variant strain IS2 (KCTC 0959BP) and the therapeutic effect for cancer disease of the glucan oligimer in any of above cited literatures, the disclosures of which are incorporated herein by reference.

The inventors of the present invention have been endeavored to find pharmacologically potent beta-glucan from specific yeast variant strain from investigate and finally completed present invention by confirming that the soluble glucan oligomer having less than 50,000 D of M.W. obtained by extracting the cell wall of yeast mutant IS2 shows potent stimulating activity of immune system and inhibiting activity of cancer cell proliferation.

These and other objects of the present invention will become apparent from the detailed disclosure of the present invention provided hereinafter.

### DISCLOSURE

According to one aspect of the present invention, the present invention provides soluble glucan oligomer derived from yeast variant strain IS2 (KCTC 0959BP) and the preparation method thereof.

Also, the present invention provides the pharmaceutical composition comprising soluble glucan oligomer prepared by the method of the present invention for promoting immunity and anti-cancer activity.

Accordingly, it is an object of the present invention to provide a soluble glucan oligomer derived from the cell wall of yeast variant strain (KTCT 0959BP) obtained by treating insoluble beta-glucan with enzyme, having immunity-promoting activity and inhibiting activity of cancer cell proliferation.

It is another object of the present invention to provide the method for preparing soluble glucan oligomer comprising the steps consisting of: (a) culturing yeast (Saccharomyces cerevisiae) variant IS2 (KCTC 0959BP) in the culture broth for inoculation; (b) inoculating above yeast culture solution to culture broth, culturing and centrifuging to obtain yeast; (c) adding NaOH thereto to extract beta-glucan from yeast cell wall; (d) reacting extracted beta-glucan with hydrolyzing enzyme and then subjecting to filtration to obtain soluble glucan oligomer; and (e) finally drying with lyophilization to obtain the soluble glucan oligomer of the present invention.

Above described yeast variant IS2 is characterized by promoting immune activity.

The soluble glucan oligomer prepared by above described procedure comprises glucan oligomer having a molecular weight of less than 50,000, preferably, ranging from 1,000 to 10,000.

It is the other object of the present invention to provide a pharmaceutical composition comprising soluble glucan oligomer derived from the cell wall of yeast variant strain (KTCT 0959BP) obtained by above described procedure as an active ingredient in an effective amount to treat and prevent immunodeficiency disease or cancer disease, together with a pharmaceutically acceptable carrier thereof.

The inventive soluble glucan oligomer may be prepared in accordance with the following preferred embodiment

For the present invention, above soluble glucan oligomer can be prepared by following procedure;

(a) 1^{st} step, the step culturing yeast IS2 (KCTC 0959BP) consisting that yeast IS2 (KCTC 0959BP) is cultured in liquid culture medium comprising 0.5-10 w/v % glucose, 0.1-5 w/v % yeast extract, 0.1-10 w/v % pepton;

(b) 2^{nd} step, the step obtaining yeast from yeast culture medium consisting that the yeast culture medium prepared from the first stage in a amount ranging from 0.1 to 10% (v/v) is inoculated to primary liquid culture medium comprising 0.5-10 w/v % glucose, 0.1-5 w/v % yeast extract, 0.01-2 w/v % ammonium sulfate, 0.001-1 w/v % potassium phosphate, and 0.001-1 w/v % magnesium sulfate in the pH ranging from 5.0 to 6.0, cultured for the period ranging from 12 hours to 48 hours at the speed ranging from 100 to 400 rpm, in the ventilating gas amount ranging from 0.3 to 3 vvm, at the temperature ranging from 20 to 40° C. in growth media and then subjected to centrifugation to obtain yeast;

(c) 3^{rd} step, the step extracting wet beta-glucan from the cell wall of the yeast consisting that 1-10% sodium hydroxide solution is added to the yeast, dispersed, reacted for the period ranging from 30 minutes to 5 hours at the temperature ranging from 70 to 100° C., subjected to centrifugation to obtain dried cell mass (DCW) of yeast, of which process may be repeated at several times to pool, titrating the pH of the mass ranging from 4.0 to 5.0 using by strong acid such as hydrochloric acid and hydrogen sulfuric acid, dispersed again in sodium hydroxide solution, further reacting for 1 hour at 75° C., subjecting centrifugation to separate to sodium hydroxide solution and solid component; and finally washing and purifying the solid component to obtain wet beta-glucan;

(d) 4^{th} step, the step obtaining liquid phase of glucan oligomer consisting that distilled water at the amount equivalent to 1 to 10 times the volume of the glucan (v/v %) and beta-glucan hydrolyzing enzyme at the amount equivalent to 1/20 to ⅕ times of the glucan (v/w %) are added thereto, reacting for the period ranging from 6 to 24 hours at the temperature ranging from 30 to 80° C., recovering supernatant solution by centrifuging after quenching the reaction, filtering supernatant with ultra filtration membrane to obtain inventive soluble glucan oligomer solution having a molecular weight of less than 50,000;

(e) 5^{th} step, the step obtaining dried powder form of final soluble glucan oligomer consisting that the oligomer prepared from 4^{th} step is left alone for the period ranging from 12 hours to 48 hours at less than −70° C., and then subject to lyophilzation to obtain the powder form of soluble glucan oligomer of the present invention.

It is the other object of the present invention to provide a process for preparing the soluble glucan oligomer as described above.

It is the other object of the present invention to provide the soluble glucan oligomer derived from yeast variant IS2 (KCTC 0959BP) prepared by the preparation as described above and it is the other object of the present invention to provide a pharmaceutical composition comprising soluble glucan oligomer derived from the cell wall of yeast variant strain (KTCT 0959BP) obtained by above described procedure as an active ingredient in an effective amount to treat and prevent immunodeficiency disease or cancer disease, together with a pharmaceutically acceptable carrier thereof.

The composition of the present invention may be used in potentiating or activating an immune system.

Above described immunodeficiency disease comprises infectious disease caused by various bacteria or virus.

Above described cancer disease comprises various disease such as lung cancer, arsenic cellular lung cancer, colon cancer, bone cancer, pancreatic cancer, skin cancer, cephalic or cervical cancer, skin or endophthalmic melanoma, hysterocarcinoma, ovarian cancer, rectal cancer, stomach cancer, perianal cancer, colonic cancer, breast cancer, endometrioma, cervical carcinoma, vaginal carcinoma, vulvul carcinoma, Hodgkin's disease, esophageal cancer, enteric cancer, endocrine gland cancer, thyroid cancer, parathyroid cancer, adrenal cancer, smooth tissue sarcoma, urethral cancer, penile cancer, prostatic cancer, chronic or acute leukemia, lymphocytoma, cystic cancer, nephritic or hydrouretic cancer, renal cell carcinoma, renal pelvic carcinoma, CNS tumor, primary CNS lymphoma, spinal medulla tumor, brain stem neuroglioma, hypophyseal adenomatosis.

In accordance with another aspect of the present invention, there provided drug combination which can add or remove appropriately another immunopotentiator or cancer drug and increase or decrease composition ratio of the drugs within the limit which can keep their efficacy.

It is an object of the present invention to provide a use of a pharmaceutical composition comprising a soluble glucan oligomer derived from yeast variant strain IS2 (KCTC 0959BP) for the preparation of therapeutic agent for treatment and prevention of immunodeficiency and cancer disease in human or mammal.

It is an object of the present invention to provide a method of treating or preventing immunodeficiency and cancer disease in a mammal comprising the step of administering to said mammal an effective amount of composition comprising a soluble glucan oligomer derived from yeast variant strain IS2 (KCTC 0959BP), together with a pharmaceutically acceptable carrier thereof.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fibers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the composition of the present invention can be dissolved in oils, propylene glycol or other solvents which are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing crude drug composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), suppository, or sterile injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active ingredients.

The desirable dose of the inventive composition varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 0.1 to 1 g/kg by weight/day of the inventive composition of the present invention. The dose may be administered in a single or multiple doses per day.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

It is still another object of the present invention to provide a health care food comprising a composition essentially comprising a soluble glucan oligomer derived from yeast variant strain IS2 (KCTC 0959BP), together with a sitologically acceptable additive for preventing and improving immunodeficiency and cancer disease.

The health care food for preventing and alleviating immunodeficiency and cancer diseases could contain about 0.01 to 80 w/w %, preferably 1 to 50 w/w % of the above soluble glucan oligomer derived from yeast variant strain IS2 (KCTC 0959BP) of present invention based on the total weight of the composition.

The present invention provides a composition of the health care food beverage for preventing and alleviating immunodeficiency and cancer disease comprising a soluble glucan oligomer derived from yeast variant strain IS2 (KCTC 0959BP).

Above inventive oligomer composition can be added to food and beverage for the preventing and alleviating immunodeficiency and cancer disease.

To develop for health care food, examples of addable food comprising above oligomer composition of the present invention are e.g., various food, beverage, bread, cookies, jam, candy, gum, tea, yogurt, vitamin complex, health improving food and the like, and can be used as power, granule, tablet, chewing tablet, capsule or beverage etc.

Inventive composition of the present invention has no toxicity and adverse effect, therefore, they can be used with safe.

Above described composition therein can be added to food, additive or beverage, wherein, the amount of above described oligomer in food or beverage may generally range from about 0.01 to 80 w/w % of total weight of food for the health care food composition and 0.02 to 30 g, preferably 0.3 to 5 g in the ratio of 100 ml of the health beverage composition.

Providing that the health beverage composition of present invention contains above described oligomer as an essential component in the indicated ratio, there is no particular limitation on the other liquid component, wherein the other component can be various deodorant or natural carbohydrate etc such as conventional beverage. Examples of aforementioned natural carbohydrate are monosacharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage.

The inventive composition can be used as the mixing agent in the lactic acid bacteria-formulated beverage or paste and the like.

Above-mentioned component can be used independently or in combination.

The present invention provides a health care food comprising about 0.01 to 30 w/w % of the vitamin, oligosaccharides and dietary ingredients besides the composition of the present invention.

The ratio of the components is not so important but is generally range from about 0.01 to 30 w/w % per 100 w/w % present composition. Examples of addable food comprising aforementioned extract therein are various food, beverage, gum, vitamin complex, health improving food and the like.

The inventive composition may additionally comprise one or more than one of organic acid, such as citric acid, fumaric acid, adipic acid, lactic acid, malic acid; phosphate, such as phosphate, sodium phosphate, potassium phosphate, acid pyrophosphate, polyphosphate; natural anti-oxidants, such as polyphenol, catechin, alpha-tocopherol, rosemary extract, vitamin C, licorice root extract, chitosan, tannic acid, phytic acid etc.

It will be apparent to those skilled in the art that various modifications and variations can be made in the compositions, use and preparations of the present invention without departing from the spirit or scope of the invention.

### DESCRIPTION OF DRAWINGS

The above and other objects, features and other advantages of the present invention will more clearly understood from the following detailed description taken in conjunction with the accompanying drawings, in which;

FIG. 1 shows the effect of soluble glucan oligomer on the NO production by mouse lymphocytes after peritoneal injection;

FIG. 2 shows the effect of soluble glucan oligomer on the IL-2 production in spleen cell when it was injected to mouse peritoneally;

FIG. 3 shows the inhibiting effect of soluble glucan oligomer on the cell proliferation of mouse bone marrow IL-3 dependent LyD9 cell line;

FIG. 4 shows the inhibiting effect of soluble glucan oligomer on the cell proliferation of Raw 264.7 cell line, a mouse macrophage cell line;

FIG. 5 shows the inhibiting effect of soluble glucan oligomer on the cell proliferation of EL4 cell line, a mouse T lymphocyte cell line;

FIG. 6 shows the inhibiting effect of soluble glucan oligomer on the cell proliferation of Jurkat cell line, a human T lymphocyte cell line;

FIG. 7 shows the inhibiting effect of soluble glean oligomer on the cell proliferation of HeLa cell line, a human cervical cancer cell line;

FIG. 8 shows the inhibiting effect of soluble glucan oligomer on the cell proliferation of KAT03 cell line, a human stomach cancer cell line.

Hereinafter, the present invention is more specifically explained by the following examples. However, it should be understood that the present invention is not limited to these examples in any manner.

### BEST MODE

The following Examples and Experimental Examples are intended to further illustrate the present invention without limiting its scope.

### Example 1

### Culture of Yeast Variant IS2 and Harvest

Liquid medium containing 10 g/L of glucose, 6 g/L yeast extract, 3 g/L of ammonium sulfate ((NH₄)SO₄), 1.5 g/L of potassium phosphate (K₂PO₄), 0.5 g/L of magnesium sulfate (MgSO₄.7H₂O) was used as primary medium.

Liquid YPD medium (glucose 20 g/L, yeast extract 10 g/L, peptone 20 g/L) was used for inoculation and growth media containing 400 g/L of glucose, 30 g/L yeast extract, 40 g/L of ammonium sulfate ((NH₄)SO₂), 15 g/L of potassium phosphate (K₂PO₄) and 5.7 g/L of magnesium sulfate (MgSO₄.7H₂O).

After autoclaving the growth media, 100 ml of cultured yeast variant IS2 (KCTC 0959BP) was seeded thereto, cultured at rotating speed of 300 rpm, at the ventilating gas amount of 1 vvm, at 30° C. pH 5.5 and finally 50-55 g/L of dried cell mass (DCW) of yeast was obtained through fed batch culture system.

### Example 2

### Extraction of Beta Glucan from Yeast Variant IS2

80 g of DCW of yeast prepared in above Example 1, was suspended in 1,000 ml of 4% sodium hydroxide (NaOH) solution and then incubated at 95° C. for 1 hour. The incubated suspension was centrifuged at the speed of 2,000 rpm for 15 minutes to separate into NaOH solution part and solid part.

The separated solid part was suspended again in 2,000 ml of 3% sodium hydroxide solution, incubated at 75° C. for 3 hours and then centrifuged at the speed of 2,000 rpm for 15 minutes to separate into NaOH solution and solid part again.

The pooled solid part was adjusted to pH 4.5 with HCl, dispersed to the extent the final volume of 2,000 ml and incubated at 75° C. for 1 hour again. The incubated suspension was centrifuged at the speed of 2,000 rpm for 15 minutes to separate into NaOH solution part and solid part.

The solid part was washed 3 times with distilled water to obtain 160 g of wet beta glucan from the cell wall of yeast variant.

### Example 3

### Preparation of Soluble Glucan Oligomer from Beta-Glucan of Yeast Variant L92

160 g of wet beta glean prepared from Example 2 was put in 1,000 ml of flask and 480 ml of distilled water and beta beta-glucanase at the amount equivalent to 1/10 of the glucan (v/w) were added thereto and incubated at 40° C. for 15 hours.

After stopping the reaction, the reaction mixture was centrifuged at 7,000 rpm for 15 minutes to collect the supernatant. The collected supernatant was filtered and the un-reacted enzymes were removed using by ultra filtration membrane (Filtron Co., MWCO 10K) to obtain the solution containing glucan oligomer having MW of less than 10,000 Dalton. After the solution had been left alone at −74° C. for overnight, the solution was lyophilized to produce 5.8 g of powder form of soluble glucan oligomer.

### Experimental Example 1

### Effect of Soluble Glucan Oligomer on NO (Nitric Oxide) Production

The soluble glucan oligomer prepared from Example 3 was dissolved in phosphate buffered saline (PBS, 2.56 g/L NaH₂PO₄.H₂O, 22.5 g/L Na HPO₄.7H₂O, 87.9 g/L NaCl, pH 7.2) at the concentration of 5 mg/ml and 0.2 ml of soluble glucan oligomer solution was peritoneally injected to 5-6 weeks aged male C57BL/6 mouse (Biolink Co.).

As a control group for the experiment, the soluble glucan oligomer derived from wild type yeast (KCTC 7911) strain was prepared in accordance with the preparation procedure described in above Example 1 to Example 3 and administrated in same manner with the method described above.

3 days after the administration, the lymphocytes was isolated softly from the spleen of above mouse using by sterilized sieve with Hank's balanced salt solution (0.185 g/L Calcium Chloride.2H₂O, 0.09767 g/L anhydrous MgSO₄, 0.4 g/L Potassium Chloride, 0.06 g/L anhydrous Potassium Phosphate Monobasic, 0.8 g/L Sodium Chloride, 0.04788 g/L anhydrous Sodium Phosphate Dibasic, 1.0 g/L D-Glucose, 0.011 g/L Phenol Red.Na, 0.35 g/L Sodium Bicarbonate, pH 7.0). Remaining RBC was removed by NH₄Cl treatment and the isolated lymphocytes were suspended in complete medium.

Peritoneal macrophage was prepared according to the method disclosed in the literature (Gallily, R., and M. Feldman., Immunology 12, p 197-, 1967).

After peritoneal injection of soluble glucan oligomer, the macrophage was collected using by Hank's balanced salt solution and then cells were suspended in complete medium.

2×10⁵ cells/ml of macrophage were resuspended at the concentration of 2×10⁵ cells/ml in cell culture plate and cultured for 20 hours under the stimulation with Concanavalin A (ConA, 5 ?g/ml) and LPS (1 ?g/ml) prior to measuring NO (nitric oxide) production in the culture medium.

Since NO is readily oxidized to stable nitrite in the air, the amount of nitrite is measured using by grease reaction.

0.1 ml of the mixture containing 1% sulfanilamide in 30% acetic acid and 0.1% N-(1-naphthyl)ethylenediamine dihydrochloride in 60% acetic add was added to 0.1 ml of culture medium and incubated at room temperature for 20 minutes. The absorbance at 550 nm was detected using by ELISA reader.

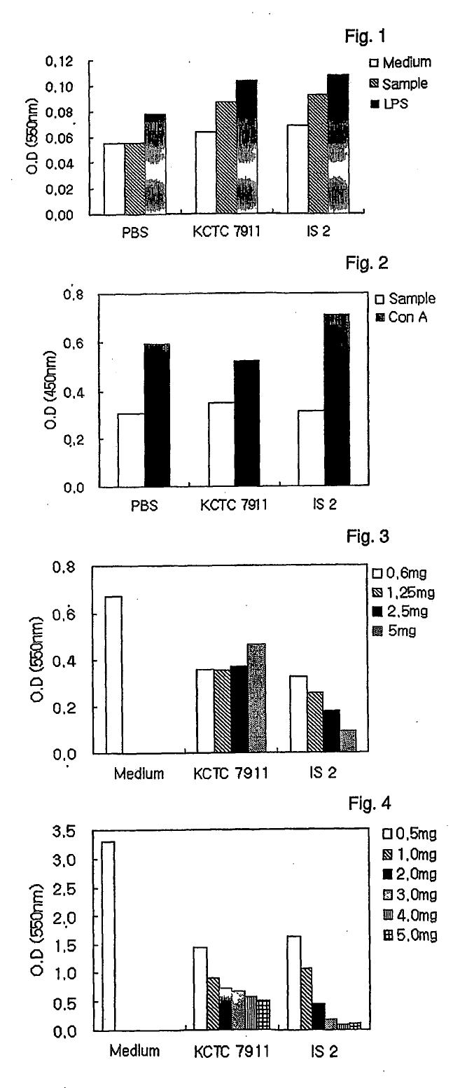

As the result of determination of NO production, the experimental groups injected with soluble glucan oligomer derived from wild yeast KTCT 7911 and yeast variant strain IS2 showed more increase of NO production compared with that of PBS-treated group as a negative control. Moreover, it was confirmed that soluble glucan oligomer derived from yeast variant strain IS2 had higher NO productivity than that from KCTC 7911 strain (See FIG. 1).

FIG. 1 shows the effect of soluble glucan oligomer on NO production by mouse lymphocytes after peritoneal injection.

### Experimental Example 2

### Effect of Soluble Glucan Oligomer on IL-2 Production

Lymphocytes were isolated from the spleen of mouse treated with soluble glucan oligomer prepared in Example 3 using sterile sieve. At that time, red blood cell was removed by NH₄Cl treatment and the isolated lymphocytes were suspended in complete medium.

Collected cells was adjusted to 2×10⁵ cells/ml and aliquoted to 96-well culture plate. Splenocytes were treated with ConA (5 ?g/ml) and incubated at 37° C. in a 5% CO₂-incubator for 48 hours. After the incubation, the amount of released IL-2 stimulated by Con-A was determined by using IL-2 ELISA kit (Endogen Co.).

Anti-IL-2 antibodies (Becton Dikinson Co.) were attached to 96 well plates for 24 hours and unattached antibodies were washed out. Subsequently, 0.1 ml of sample was added thereto and incubated for 1 hour. And then each well was washed with PBS containing 10% Tween-20 and the detection Ab therefor was added thereto. After washing again, streptavidin-HRP was treated and incubated at room temperature for 1 hour. TMB substrate (Becton Dikinson Co.) was added thereto and stop solution was also added for stopping the reaction. Absorbance was measured at 450 nm.

Where the soluble glucan oligomer derived from KCTC 7911 was administered, there was no change of IL-2 production, even when cells were stimulated by Con-A.

However, where the soluble glucan oligomer derived from yeast variant IS2 was administered and the cells were stimulated with ConA, it showed increased production of IL-2 from peritoneal macrophages (See FIG. 2).

FIG. 2 shows the effect of soluble glucan oligomer on IL-2 production in spleen cell when it is peritoneally injected to mouse.

### Experimental Example 3

### The inhibitory effect on cancer cell proliferation of soluble glucan oligomer

The inhibitory effect on the growth of the cancer cell lines was determined by MTT(3-[4,5-dimethylthiazol-2-yl]-2,5-diphenyltetrazolium bromide) cell proliferation assay.

As cell lines for the present experiment, 6 types of cell lines were used in this experiment, i.e., LyD9 cell line which is mouse bone marrow IL-3 dependent hematopoietic stem cell line, Raw264.7 cell line which is mouse macrophage cell line, EL4 cell line which is mouse T lymphoma cell line, Jurkat cell line which is human T lymphoma cell line, HeLa cell line which is human cervical carcinoma cell line and KAT03 cell line which is human stomach cancer cell line.

The cell was treated with samples at the indicated concentrations, incubated for 44 hours and MTT solution was added thereto for determination of cell viability.

Cancer cells were adjusted to the concentration of 2×10⁵ cells/ml and transferred to each well of 96-well plate. In early culture, soluble glucan oligomer was added to the medium with various concentrations ranging from 0.5 to 5 mg/ml and the culture medium was further cultured at 37° C. in a 5% CO₂-incubator for 48 hours.

The cell viability was measured using MTT assay.

MTT (Sigma Co., USA) reagent was added into each well and it was further cultured for 4 hours. After adding 100 ml of 0.04N HCl in isopropanol thereto, the mixture was incubated at room temperature for 20 minutes and finally absorbance was determined at 550 nm by using ELISA reader.

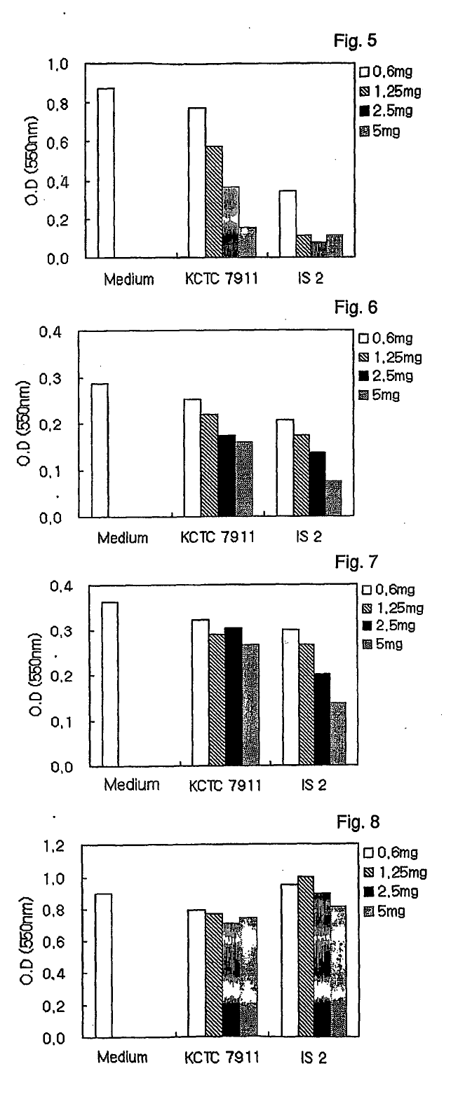

As can be seen in FIG. 3 to 8, the soluble glucan oligomer of the present invention showed potent the inhibiting activity for the proliferation of cancer cell. In particular, the soluble glucan oligomer derived from yeast variant IS2 showed more potent activity than that from KCTC 7911, and specifically, the soluble glen oligomer derived from yeast variant IS2 showed about 2 times to 6.6 times of potency compared with that from KCTC 7911 at the same concentration. It is confirmed that effective concentration of the soluble glucan oligomer ranges from 2.5 to 5.0 mg in most of cancer cell lines.

### Experimental Example 4

### Toxicity Test

Methods

The acute toxicity tests on ICR mice (mean body weight 25±5 g) and Sprague-Dawley rats (235±10 g, Jung-Ang Lab Animal Inc.) were performed using the oligomer of the Example 3. Four group consisting of 10 mice or rats was administrated orally with 4 mg/kg, 40 mg/kg, 400 mg/kg and 4,000 mg/kg of test sample or solvents (0.2 ml, i.p.) respectively and observed for 2 weeks.

Results

There were no treatment-related effects on mortality, clinical signs, body weight changes and gross findings in any group or either gender. These results suggested that the extract prepared in the present invention were potent and safe.

Hereinafter, the formulating methods and kinds of excipients will be described, but the present invention is not limited to them. The representative preparation examples were described as follows.

Preparation of powder

| | |
|---|---|
| Dried powder of Example 3 | 50 mg |
| Lactose | 100 mg |
| Talc | 10 mg |

Powder preparation was prepared by mixing above components and filling sealed package.

Preparation of tablet

| | |
|---|---|
| Dried powder of Example 3 | 50 mg |
| Corn Starch | 100 mg |
| Lactose | 100 mg |
| Magnesium Stearate | 2 mg |

Tablet preparation was prepared by mixing above components and entabletting.

Preparation of capsule

| | |
|---|---|
| Dried powder of Example 3 | 50 mg |
| Corn starch | 100 mg |
| Lactose | 100 mg |
| Magnesium Stearate | 2 mg |

Tablet preparation was prepared by mixing above components and filling gelatin capsule by conventional gelatin preparation method.

Preparation of injection

| | |
|---|---|
| Dried powder of Example 3 | 50 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of liquid

| Dried powder of Example 3 | 0.1˜80 g |
|---|---|
| Sugar | 5˜10 g |
| Citric acid | 0.05˜0.3% |
| Caramel | 0.005˜0.02% |
| Vitamin C | 0.1˜1 % |
| Distilled water | 79˜94% |
| CO₂ gas | 0.5˜0.82% |

Liquid preparation was prepared by dissolving active component, filling all the components and sterilizing by conventional liquid preparation method.

Preparation of health care food

| | |
|---|---|
| Dried powder of Example 3 | 1000 mg |
| Vitamin mixture | optimum amount |
| ? Vitamin A acetate | 70 mg |
| ? Vitamin E | 1.0 mg |
| ? Vitamin B₁ | 0.13 mg |
| ? Vitamin B₂ | 0.15 mg |
| ? Vitamin B₆ | 0.5 mg |
| ? Vitamin B₁₂ | 0.2 mg |
| ? Vitamin C | 10 mg |
| ? Biotin | 10 mg |
| ? Amide nicotinic acid | 1.7 mg |
| ? Folic acid | 50 mg |
| ? Calcium pantothenic acid | 0.5 mg |
| Mineral mixture | optimum amount |
| ? Ferrous sulfate | 1.75 mg |
| ? Zinc oxide | 0.82 mg |
| ? Magnesium carbonate | 25.3 mg |
| ? Monopotassium phosphate | 15 mg |
| Dicalcium phosphate | 55 mg |
| Potassium citrate | 90 mg |
| Calcium carbonate | 100 mg |
| Magnesium chloride | 24.8 mg |

The above-mentioned vitamin and mineral mixture may be varied in many ways. Such variations are not to be regarded as a departure from the spirit and scope of the present invention.

Preparation of health beverage

| | | |
|---|---|---|
| Dried powder of Example 3 | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the detailed description of the present invention, the soluble glucan oligomer having a M.W. ranging from 1,000 to 10,000 prepared by treating insoluble beta-glucan isolated from the cell wall of yeast variant IS2 with commercially available beta-glucan hydrolyzing enzymes, showed potent efficacy on promoting immune activity and on inhibiting the growth of cancer cell, therefore, it can be used as the therapeutics or health care food for treating and preventing immunodeficiency and cancer disease.

1. A soluble glucan oligomer derived from the cell wall of yeast variant strain (KTCT 0959BP) obtained by treating insoluble beta-glucan with hydrolyzing enzyme, having immunity-promoting activity and inhibiting activity of cancer cell proliferation.

2. A method for preparing soluble glucan oligomer comprising the steps consisting of: (a) culturing yeast (Saccharomyces cerevisiae) variant IS2 (KCTC 0959BP) in the culture broth for inoculation; (b) inoculating above yeast culture solution to culture broth, feed batch culturing and centrifuging to obtain yeast; (c) adding NaOH thereto to extract beta-glucan from yeast cell wall; (d) reacting extracted beta-glucan with hydrolyzing enzyme and then subjecting to filtration to obtain soluble glucan oligomer; and (e) finally drying with lyophilization to obtain the soluble glucan oligomer having a molecular weight of less than 50,000.

3. A pharmaceutical composition comprising the soluble glucan oligomer derived from the cell wall of yeast variant strain (KTCT 0959BP) obtained by the preparation method as set forth in claim 2, as an active ingredients for the treatment and prevention of immunodeficiency disease.

4. A pharmaceutical composition comprising the soluble glucan oligomer derived from the cell wall of yeast variant strain (KTCT 0959BP) obtained by the preparation method as set forth in claim 2, as an active ingredients for the treatment and prevention of cancer disease.

5. The pharmaceutical composition according to claim 4 wherein said cancer is selected from the group consisting of lung cancer, arsenic cellular lung cancer, colon cancer, bone cancer, pancreatic cancer, skin cancer, cephalic or cervical cancer, skin or endophthalmic melanoma, hysterocarcinoma, ovarian cancer, rectal cancer, stomach cancer, perianal cancer, colonic cancer, breast cancer, endometrioma, cervical carcinoma, vaginal carcinoma, vulvul carcinoma, Hodgkin's disease, esophageal cancer, enteric cancer, endocrine gland cancer, thyroid cancer, parathyroid cancer, adrenal cancer, smooth tissue sarcoma, urethral cancer, penile cancer, prostatic cancer, chronic or acute leukemia, lymphocytoma, cystic cancer, nephritic or hydrouretic cancer, renal cell carcinoma, renal pelvic carcinoma, CNS tumor, primary CNS lymphoma, spinal medulla tumor, brain stem neuroglioma, and hypophyseal adenomatosis.

6. A use of a pharmaceutical composition comprising a soluble glucan oligomer derived from yeast variant strain IS2 (KCTC 0959BP) for the preparation of therapeutic agent for treatment and prevention of immunodeficiency and cancer disease in human or mammal.

7. A healthcare food comprising a soluble glucan oligomer derived from yeast variant strain IS2 (KCTC 0959BP) as set forth in claim 1, together with asitologically acceptable additive for the prevention and improvement of immunodeficiency and cancer disease.

8. The health care food according to claim 7 wherein said health care food is provided as powder, granule, tablet, chewing tablet, capsule or beverage type.
